(19) United States
(12) Patent Application Publication
(10) Pub. No.: US 2011/0224639 A1
(43) Pub. Date: Sep. 15, 2011

(54) Absorbent Garment

(76) Inventor: Sateria VENABLE, Chicago, IL (US)

(21) Appl. No.: 12/721,656

(22) Filed: Mar. 11, 2010

(51) Int. Cl. A61F 13/45

(52) U.S. Cl. 604/385.01

(57) ABSTRACT

An absorbent garment is provided having increased surface area of absorbent material as compared to conventional absorbent garments, such as sanitary napkins, diapers, and the like. In one aspect, the absorbent garment provides increased surface area around the inner thigh area of the wearer. Such increased surface area of absorbent material of the garments described herein provides increased protection of leakage from the garment during sleeping, sports, normal daily wear, and other physical activities.

### TECHNICAL FIELD

This invention relates generally to absorbent personal care garments.

### BACKGROUND

Numerous personal care products are on the market today that are designed to absorb bodily fluids. Such absorbent products include diapers, sanitary napkins, incontinence briefs, and panty-liners. These products are often sold in different thicknesses to provide different levels of absorbency. These products are configured to typically collect bodily fluids directly adjacent the source of the flow of the bodily fluids.

Sanitary napkins have been available for many years. Sanitary napkins are typically removably attached via adhesive to the inside of the panties or undergarments of the user. Sanitary napkins with tabs or “wings” are designed to be placed in the user's undergarments with the tabs wrapped around the edges and outside surface of the undergarment. These sanitary napkins are usually kept in place due to an adhesive on the exterior surface of the sanitary napkin and tabs.

The sanitary napkins as known in the art often shift during wearing, such as during sports or while sleeping. The products also frequently deform in shape during usage, which can reduce the effectiveness of the napkin in preventing leakage. Shifting and deformation in shape of the napkin can also cause flow channels to form in the napkin, which can result in leakage of bodily fluids and staining of the wearer's undergarments, clothing, and/or support surfaces such as furniture or the like.

Sanitary napkins are offered in a variety of thicknesses for absorbing different levels of menstrual flow. The thicker, extra-absorbent varieties of these products which are offered for heavy flow can be unduly bulky and cumbersome to the wearer. Indeed, even these products designed to accommodate heavy flow often fail to prevent leakage upon heavy flow. The applicant has determined that certain medical conditions, such as fibroids, can cause heavy menstrual flow or “gushing,” which can leak around the sides and ends of the napkin when the flow is too fast and/or the quantity of flow too great to be absorbed by the napkin before leaking around the side(s) and end(s) of the article.

The applicant has further determined that even medical-grade sanitary napkins, used frequently in hospitals, can and do shift during wear and can be cumbersome as well to the wearer. Generally, commercially-available sanitary products presume, and are designed to accommodate, a slow, steady flow of menstrual blood, urine, or bodily fluids, not a sudden, larger quantity.

The failure of these articles often leaves the wearer with embarrassing leakage and staining of outer garments. This, in turn, can lead to any number of behaviors and/or reactions that are, at the least, socially and economically disadvantageous and, perhaps at the worst, injurious to self esteem and/or an actual or perceived inability to assume or to maintain gainful employment.

Many sanitary napkins are available with “wings” or “tabs” that extend from the sides of the napkin and wrap around the wearer's underwear so as protect the edges of the panty from leakage from the sides of the sanitary napkin. The applicant, however, has determined that such wings or tabs have significantly reduced absorbency as compared to the remainder of the article. It is also problematic from a leakage perspective because adhering the wings or tabs to the outer crotch area of the undergarment can cause the overall shape of the napkin crotch region to slope towards the sides of the wearer's underwear, thereby actually increasing the likelihood of leakage as the sides of the napkin gap away from the body.

The applicant has further determined that such wings also often fail to protect the wearer from bodily fluids that skim the surface of the sanitary napkin and leak from the sides and sometimes the ends of the napkin, only to be absorbed by the wearer's clothing. Wings also sometimes fail to protect the wearer from leakage that results when the napkin is distorted, twists, or bends during normal wear, thereby causing fluid to not be adequately captured by the napkin.

At least one prior art approach attempts to address this problem through the use of barrier structures on the tabs. By one approach, such a sanitary napkin uses side-barrier devices which project upwardly from the surface of the positioning tab formed along the interface between the tab and the main body portion of the sanitary napkin. Such side-barrier devices, however, typically require that the sanitary napkin always be held firmly to the skin, such that any liquid cannot pass by the side barrier devices. Notwithstanding the helpful intent of such an approach, the applicant has determined that such ideal conditions are simply not realistic and the approach can fail in the field for a variety of reasons.

Absorbent garments are therefore faced with a variety of utilitarian design requirements in these regards, many of which tend to be contrary to one another. The market for such products requires highly absorbent garments but which are also not bulky and uncomfortable to the wearer.

### BRIEF DESCRIPTION OF THE DRAWINGS

The above needs are at least partially met through provision of the absorbent garments described in the following detailed description, particularly when studied in conjunction with the drawings, wherein:

FIGS. 1(A) and 1(B) comprise a front view of an absorbent garment as configured in accordance with various embodiments of the invention;

FIG. 2 comprises a side view of an absorbent garment as configured in accordance with various embodiments of the invention;

FIG. 3 comprises a top view of an absorbent garment as configured in accordance with various embodiments of the invention;

FIG. 4 comprises a sectional view of an absorbent garment along line x-x of the absorbent garment of FIG. 3 as configured in accordance with various embodiments of the invention;

FIG. 5 comprises a sectional view of an absorbent garment along line x-x of the absorbent garment of FIG. 6 as configured in accordance with various embodiments of the invention;

FIG. 6 comprises a top view of an absorbent garment as configured in accordance with various embodiments of the invention;

FIG. 7(A) comprises a top view of an absorbent garment as configured in accordance with various embodiments of the invention and FIG. 7(B) comprises a sectional view along line x-x of the absorbent garment of FIG. 7(A);

FIG. 8(A) comprises a top view of an absorbent garment as configured in accordance with various embodiments of the invention and FIG. 8(B) comprises a sectional view along line x-x of the absorbent garment of FIG. 8(A);

FIGS. 9(A), 9(B), and 9(C) each comprise a sectional view of an absorbent portion of an absorbent garment as configured in accordance with various embodiments of the invention;

FIGS. 10(A) and 10(B) each comprise a sectional view of an absorbent garment along line x-x of the absorbent garment of FIG. 6 as configured in accordance with various embodiments of the invention;

FIGS. 11(A), 11(B), and 11(C) show component parts of an absorbent portion of an absorbent garment as configured in accordance with various embodiments of the invention; and

FIG. 12 shows component parts of an absorbent garment as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the invention. Also, common but well-understood elements that are useful or necessary in a commercially feasible embodiment are often not depicted in order to facilitate a less obstructed view of these various embodiments of the invention. It will further be appreciated that certain actions and/or steps may be described or be depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above (except where different specific meanings have otherwise been set forth herein).

### DETAILED DESCRIPTION

Generally speaking, pursuant to these various embodiments, an absorbent garment is provided having increased surface area of absorbent material as compared to conventional absorbent garments, such as sanitary napkins, diapers, and the like. The absorbent garment provides increased protection to the wearer against leakage of bodily fluids and prevents soiling of the wearer's outer garments, while also wicking moisture from the body.

Such increased surface area of absorbent material of the garments described herein provides increased protection against leakage from the garment during daily wear, working, sleeping, sports, and other physical activities. These approaches also provide increased protection against leakage that might otherwise occur due to unusually high-volume flows. Such benefits compare in great contrast to conventional personal hygiene products that typically suffer from leakage both laterally and longitudinally from the crotch portion of the garment. These teachings therefore greatly assist with avoiding embarrassing leakage and corresponding dampening and staining of the wearer's other garments.

By one approach, the absorbent garment is provided in a boy-short format. As used herein, a “boy-short” is a type of undergarment which, unlike traditional panties or briefs, covers the entire buttocks region and part of the upper thigh area of the wearer (typically in a body-shape conformal manner). In one aspect, the absorbent garment described herein includes absorbent material provided both along the upper thigh region of each leg as well as the crotch portion of the garment. In another aspect, the absorbent garment described herein also includes absorbent material forward from and to the rear of the crotch region.

The absorbent garments described herein provide increased surface area of absorbent material (particularly as presented to and in contact with the wearer's skin) as compared to a conventional sanitary napkin with wings, particularly in the thigh area of the wearer. By providing such increased surface area of absorbent material, the garment described herein provides increased ability of the garment to avoid leakage under a variety of challenging operating conditions.

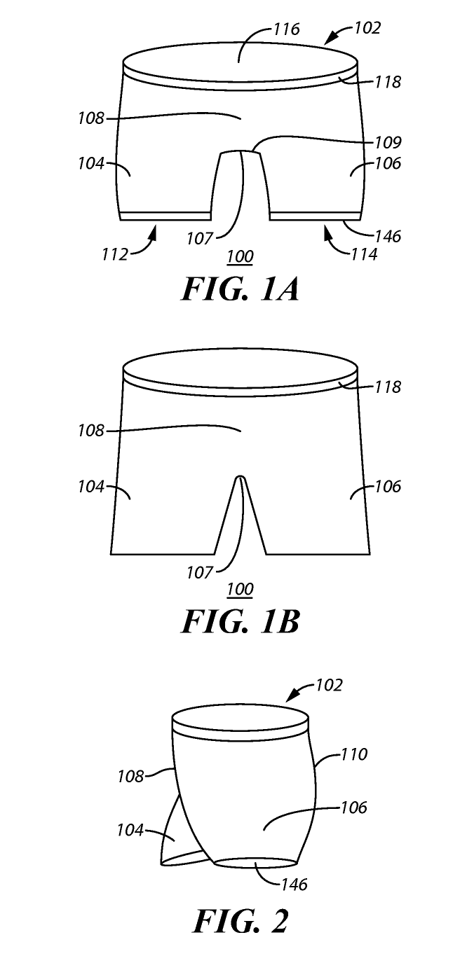

These and other benefits may become clearer upon making a thorough review and study of the following detailed description. Referring now to the drawings, and in particular to FIGS. 1(A)-(B) and 2, an absorbent garment 100 is provided that includes a waist portion 102, two leg portions 104 and 106, a crotch portion 107, a front torso portion 108 which, when worn, is positioned on the front of the wearer, and a back torso portion 110 (shown in FIG. 2) which, when worn, is positioned on the back of the wearer.

In one aspect, the crotch portion 107 is located adjacent the flow source when the garment is worn by the user. By one optional but preferred approach and as shown in FIG. 1(A), the crotch portion 107 can be provided in the form of a gusset 109 as is sometimes used in the manufacture of undergarments, such as pantyhose or tights. The term “gusset” refers to an area or piece of fabric, generally in the shape of a diamond, rectangle, or triangle (although the particular shape is not critical), in the crotch portion which allows for more fabric on a relatively horizontal plane to provide improved fit of the crotch region to the wearer. FIG. 1(B) depicts a non-gusseted absorbent garment 100. Other configurations may also be used to provide for more fabric on a relatively horizontal plane adjacent the flow source when worn by the wearer.

The term “fabric” refers to all woven, knitted, and nonwoven fibrous webs. Nonwoven materials are those which are formed without the aid of a textile weaving or knitting process. The term “portion” when used in the singular herein can have the dual meaning of a single element or a plurality of elements.

The leg portions 104 and 106 extend downwardly from front and back torso portions 108 and 110 and are connected by the crotch portion 107. The lower end of each leg portion includes an opening (denoted in FIG. 1(A) by reference numerals 112 and 114) sized to permit a leg to pass therethrough. The waist portion 102 includes a waist opening 116 and a waist band 118 around the perimeter of the waist opening 116. The portions of the garment referred to herein may comprise a single piece of fabric or two or more pieces of fabric operatively associated together to form the garment.

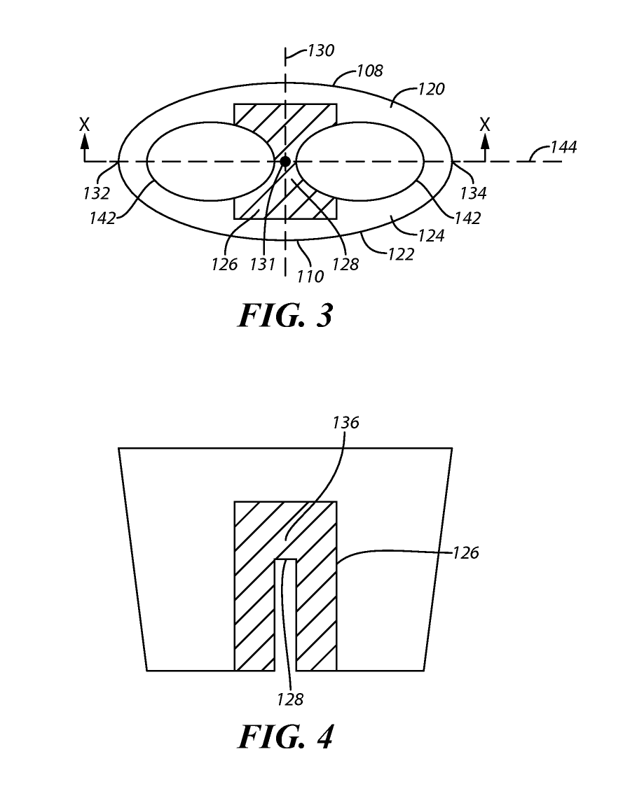

As shown in FIG. 3, the absorbent garment 100 further includes an interior portion 120 and an exterior portion 122. The interior portion 120 is configured to contact the wearer's skin while the exterior portion 122 faces the wearer's clothing and is opposite the interior portion 120. In one aspect and as shown in FIG. 3, the interior portion 120 comprises at least one non-absorbent portion 124 and at least one absorbent portion 126. As used herein, “absorbent” refers to material(s) capable of absorbing and containing bodily fluids, secretions, and exudates discharged from the body in amounts in excess of the absorbent material's own mass. As also used herein, “non-absorbent” refers to material(s) which are not known to those skilled in the art for their capacity for absorbing and containing bodily fluids, secretions, and exudates discharged from the body.

The absorbent portion 126 includes a central absorbent region 128 adjacent the fluid flow source (i.e., the crotch region of the garment). The configuration of the central absorbent region 128 is such that the majority of the liquid from the flow source is collected and absorbed by the central absorbent region. The central absorbent region is oriented about a longitudinal center line 130. A center point 131 of the central absorbent region is positioned at the intersection of this longitudinal center line 130 and a latitudinal line 144. The center point 131 is the centermost point of the central absorbent region 128. The longitudinal line 130 bisects the garment into a left portion 132 and a right portion 134. The latitudinal line 144 bisects the garment into a corresponding front torso portion 108 and a back torso portion 110.

In one aspect, the central absorbent region 128 has a width along latitudinal line 144 of at least about 1 inch. In another aspect, the central absorbent region 128 has a width along latitudinal line 144 of at least about 2 inches.

By one approach, and as shown in FIG. 4, the absorbent portion 126 can include a forward absorbent region 136 and a rear absorbent region (not shown) opposite the front absorbent portion, both of which extend upwardly from the central region 128 (i.e., the forward absorbent region 136 extends upwardly toward the wearer's abdomen and the rear absorbent region extends upwardly toward the wearer's lower back). In one aspect, the forward absorbent region 136 and rear absorbent region can be considered part of or extensions of the central absorbent region 128.

The length of the central absorbent region and/or forward absorbent region and rear absorbent region can vary depending on the particular needs of the wearer. For instance, absorbent garments worn while sleeping may have a longer area of absorbent region located to the rear of the garment. In one aspect, the central absorbent region (including any forward and/or rear absorbent regions) extends at least about 2 inches forward and/or rear of the center point 131 of the central absorbent region 128. In another aspect, the central absorbent region (including any forward and/or rear absorbent regions) extends at least about 3 inches forward and/or rear of the center point 131 of the central absorbent region 128. In another aspect, the central absorbent region (including any forward and/or rear absorbent regions) extends at least about 4 inches forward and/or rear of the center point 131 of the central absorbent region 128. In another aspect, the central absorbent region (including any forward and/or rear absorbent regions) extends at least about 5 inches forward and/or rear of the center point 131 of the central absorbent region 128. In another aspect, the central absorbent region (including any forward and/or rear absorbent regions) extends at least about 6 inches forward and/or rear of the center point 131 of the central absorbent region 128.

The center point 131 of the central absorbent region 128 is generally configured to be adjacent the flow source when the undergarment is being worn. For example, the center point 131 of the central absorbent region 128 is shown in FIG. 3. In another aspect, the central absorbent region (including any forward and/or rear absorbent regions) extends about 2 to about 10 inches forward and/or rear of the center point 131 of the central absorbent region 128. In another aspect, the central absorbent region (including any forward and/or rear absorbent regions) extends about 3 to about 10 inches forward and/or rear of the center point 131 of the central absorbent region 128. In another aspect, the central absorbent region (including any forward and/or rear absorbent regions) extends about 4 to about 10 inches forward and/or rear of the center point 131 of the central absorbent region 128. In yet another aspect, the central absorbent region (including any forward and/or rear absorbent regions) extends about 5 to about 10 inches forward and/or rear of the center point 131 of the central absorbent region 128.

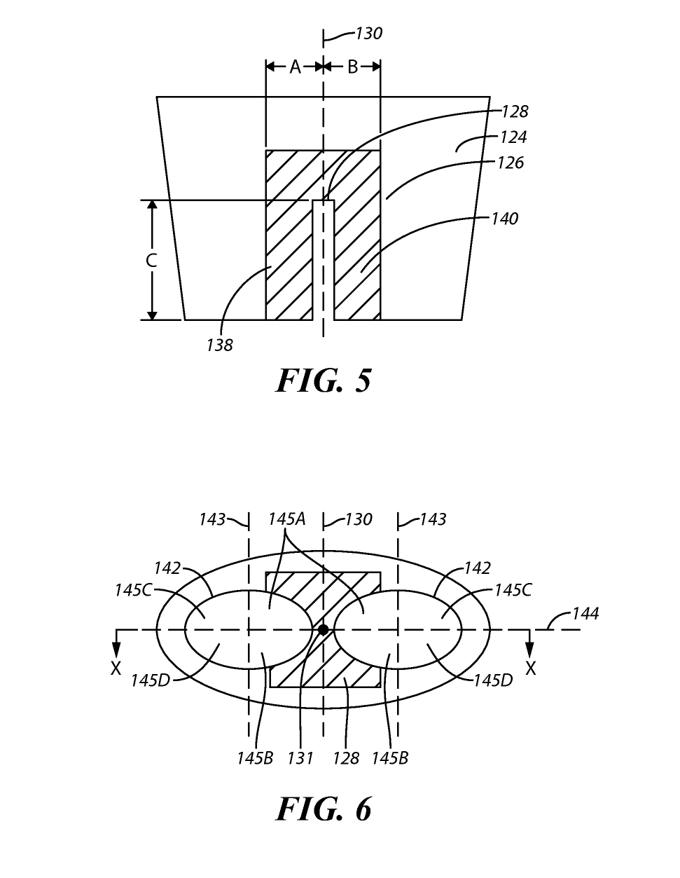

Similarly, the width of the central absorbent region, as well as any forward and rear absorbent regions, may vary depending on the specific needs of the wearer. FIG. 5 depicts an absorbent garment having the longitudinal line 130 bisecting the forward absorbent region 136. As depicted therein, the width of the forward absorbent region 136 is provided by a length “A” and length “B” on either side of the longitudinal line 130. The same teaching applies to the rear absorbent region.

Lengths “A” and “B” may be the same or different although generally distances “A” and “B” are the same for any number of useful application settings. Any desired length “A” or “B” may be used depending on the needs of the wearer but is generally between about 1 to about 10 inches. In one aspect, length “A” or “B” is between about 1 to about 8 inches. In one aspect, length “A” or “B” is between about 1 to about 5 inches. In another aspect, length “A” or “B” is between about 2 to about 5 inches. In yet another aspect, length “A” or “B” is between about 3 to about 5 inches. If lengths “A” or “B” are the same, the total width of the forward or rear absorbent region is twice the length of “A.” The length “A” or “B” may also change based on proximity to the flow source. For example, the length of “A” or “B” may be longer closer to the center absorbent region and become shorter as the distance from the center absorbent region increases. In yet another aspect, absorbent material may be incorporated around the entire circumference of the absorbent garment, if desired.

The dimensions provided herein are intended to provide exemplary dimensions for an absorbent garment but are not intended to be limiting because the various dimensions may also depend on the weight, size, and shape of the wearer, as well as more specific needs of that wearer. The dimensions for a particular wearer can be readily selected by one of ordinary skill in the art based on the teachings herein. The dimensions will also be dictated, at least in part, by manufacturing considerations, including cost and ease of machinability, as well as in manufacturing various sizes of the absorbent garment.

In another aspect, as shown in FIG. 5, two absorbent thigh regions 138 and 140 extend downwardly from the central absorbent region 128 and are generally symmetrically oriented about the center region 128 such that the two thigh regions are substantially mirror images of each other, although asymmetrical configurations may be provided as well depending on the needs of the particular user. The absorbent thigh regions and forward and rear absorbent regions are intended to collect and absorb the fluids not collected by or that have spilled over from the central absorbent region.

The absorbent thigh regions 112 and 114 extend a length “C” downwardly from the central absorbent region 128 of the crotch portion 107 of the garment 100. In one aspect, length “C” is at least about 1 inch. In another aspect, length “C” is at least about 1.5 inches. In another aspect, length “C” is at least about 2 inches. In another aspect, length “C” is at least about 3 inches. In another aspect, length “C” is at least about 4 inches. In another aspect, length “C” is at least about 5 inches. In yet another aspect, length “C” of the thigh regions 112 and 114 extend about 2 to about 7 inches downwardly from the central region 128 of the garment 100. In yet another aspect, length “C” of the thigh regions 112 and 114 extend about 3 to about 5 inches downwardly from the central region 128 of the garment 100. The absorbent thigh regions 112 and 114 advantageously provide absorbent material for capturing and retaining bodily fluids and exudates which are not retained by the central absorbent region 128.

As discussed above, while the length of the absorbent thigh regions may vary, the amount of absorbent material provided around the circumference of the thigh can also vary to provide the desired absorbency. Referring again to FIG. 3, the leg portions 104 and 106 each are provided with an approximately circular shaped (i.e., generally conforming to the shape of a wearer's leg) interior having a perimeter 142 configured for positioning a leg of the wearer therethrough.

As shown in FIG. 6, the perimeter 142 of each leg portion has a circumference of 360 degrees. The approximately circular interior of each leg portion is divided by a longitudinal line 143 that is parallel to the longitudinal line 130 of the central region 128. The approximately circular interior of each leg portion is also divided by the latitudinal line 144 that is perpendicular to the longitudinal lines 143. The longitudinal lines 143 and latitudinal line 144 thus bisect each leg portion into four 90 degree quadrants: quadrants 145A-D.

Absorbent material may be provided around a portion of some or all of the four quadrants to provide absorbent thigh regions 138 and 140, depending in part on the absorbency needs of the particular user. Quadrants 145A and 145B are located adjacent the wearer's inner thigh when the garment is worn while quadrants 145C and 145D are located opposite the wearer's inner thigh when the garment is worn (i.e., the area of thigh below the wearer's hips). For example, for light flow, a wearer may only require or desire absorbent material positioned in some or all of quadrants 145A and 145B while absorbent material positioned in quadrants 145C and 145D may be superfluous.

Similarly, in cases of heavy flow, a wearer may require or desire absorbent material in all of quadrants 145A and 145B as well as in some or all of quadrants 145C and 145D. Specific examples in this regard are described in more detail in reference to FIGS. 6, 7(A) and 7(B), and 8(A) and 8(B). In FIG. 6, for example, the thigh portions adjacent quadrants A and B are partially lined (approximately 65 degrees in either direction along the depicted latitudinal line 144) with absorbent material while no absorbent material is provided adjacent quadrants 145 C and 145D. This configuration provides approximately 130 total degrees of absorbent material (i.e., about 65 degrees from each of quadrants 145A and B) adjacent each inner thigh of the wearer with respect to the absorbent thigh regions 138 and 140.

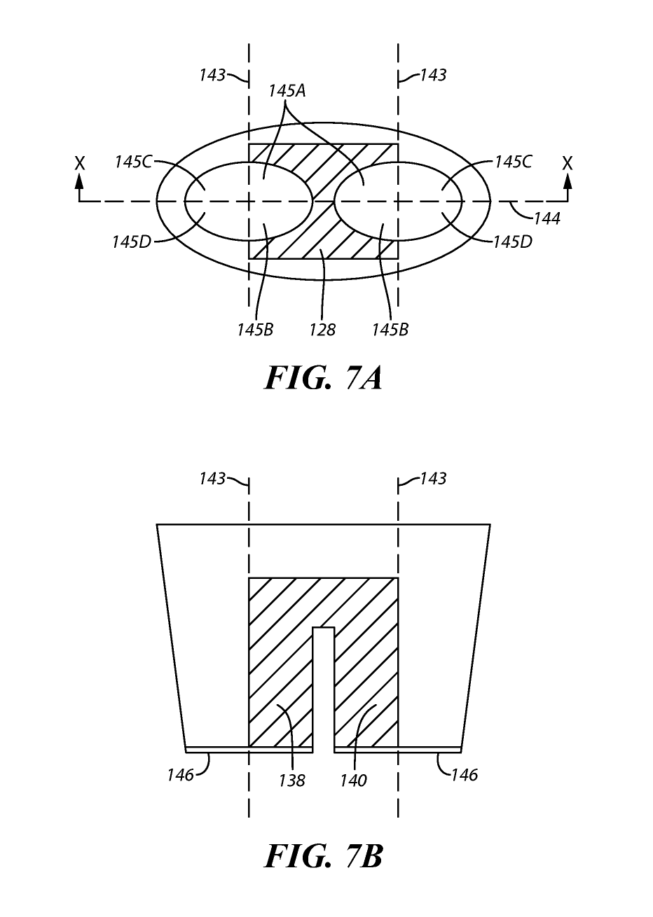

In another aspect and as shown in FIGS. 7(A) and 7(B), the thigh portions adjacent quadrants 145A and 145B are lined with absorbent material while no absorbent material is provided adjacent quadrants 145C and 145D. This configuration provides 180 degrees of absorbent material (i.e., 90 degrees from quadrant 145A and 90 degrees from quadrant 145B) adjacent each inner thigh of the wearer with respect to the absorbent thigh regions 138 and 140.

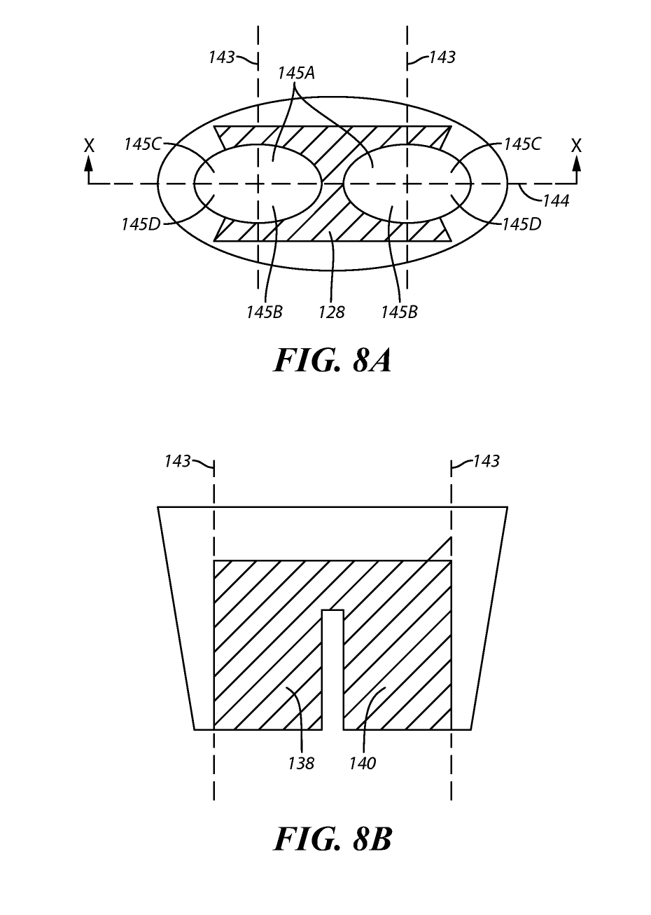

In yet another aspect and as shown in FIGS. 8(A) and 8(B), the thigh portions adjacent quadrants 145A and 145B are lined with absorbent material and the thigh portions adjacent quadrants 145C and 145D are partially lined. This configuration provides about 270 degrees of absorbent material (i.e., about ninety degrees each from quadrants 145A and B, and about 45 degrees each from quadrants 145C and D) for each inner thigh of the wearer.

While not pictured herein, it is also contemplated that absorbent material can be provided substantially around quadrants 145A-D to provide 360 degrees of absorbent material for each thigh of the wearer.

A variety of configurations in this respect are contemplated herein. Generally, however, it is desirable to provide at least about 90 total degrees of absorbent material (i.e., about 45 degrees from each of quadrants 145A and B) adjacent each inner thigh of the wearer with respect to absorbent thigh regions 138 and 140. In one aspect, about 90 to about 270 degrees of absorbent material is configured to be positioned adjacent each inner thigh of the wearer. In another aspect, about 90 to about 230 degrees of absorbent material is configured to be positioned adjacent each inner thigh of the wearer. In another aspect, about 90 to about 180 degrees of absorbent material is configured to be positioned adjacent each inner thigh of the wearer. In yet another aspect, about 90 to about 130 degrees of absorbent material is configured to be positioned adjacent each inner thigh of the wearer. These configurations are generally effective for light incontinence and menses.

Heavy incontinence and menses sometimes require a greater minimum degree of absorbent material, such as at least about 180 degrees of absorbent material configured to be positioned adjacent each inner thigh of the wearer. In one aspect, about 180 degrees to about 360 degrees of absorbent material per thigh is preferred in this respect. In other words, the absorbent thigh portions may completely encircle the wearer's upper thighs, if desired. In another aspect, about 180 to about 270 degrees of absorbent material per thigh can be provided. For even heavier flow, about 270 to about 360 degrees of absorbent material per thigh can be provided.

The absorbent garments described herein provide increased surface area of absorbent material as compared to a conventional sanitary napkin with wings, particularly in the thigh area of the wearer, to provide increased ability of the garment to avoid leakage. In one aspect, the combined surface area of the absorbent thigh regions provide at least as much surface area of absorbent material as that provided by the central absorbent region (including any forward and rear absorbent region). In another aspect, the combined surface area of the absorbent thigh regions provide at least 20 percent more surface area of absorbent material as that provided by the central absorbent region (including any forward and rear absorbent region). In another aspect, the combined surface area of the absorbent thigh regions provide at least 30 percent more surface area of absorbent material as that provided by the central absorbent region (including any forward and rear absorbent region). In another aspect, the combined surface area of the absorbent thigh regions provide at least 40 percent more surface area of absorbent material as that provided by the central absorbent region (including any forward and rear absorbent region). In another aspect, the combined surface area of the absorbent thigh regions provide 50 percent more surface area of absorbent material as that provided by the central absorbent region (including any forward and rear absorbent region). In another aspect, the combined surface area of the absorbent thigh regions provide 60 percent more surface area of absorbent material as that provided by the central absorbent region (including any forward and rear absorbent region). In yet another aspect, the combined surface area of the absorbent thigh regions provide 75 percent more surface area of absorbent material as that provided by the central absorbent region (including any forward and rear absorbent region). Even higher ratios of surface area of the absorbent thigh regions to the central absorbent region can be provided if desired.

Other configurations of the absorbent thigh regions may be provided, if desired, and may depend in part on the level of flow desired to be contained. Generally, a desire to absorb a larger flow may require a larger coverage of absorbent material, such as in the thigh region.

The leg portions 104 and 106 should fit snugly but comfortably against the wearer's body so as to maintain substantially continuous contact between the wearer's leg and the absorbent thigh regions 112 and 114 regardless of whether the wearer is in repose or physically active. Such substantially continuous contact helps to ensure that any liquid not absorbed by the central region 128 will come into contact with and be absorbed by the absorbent material of the thigh regions 112 and 114. If desired, the leg portions 104 and 106 can be provided with leg bands 146 to reduce or prevent movement of the thigh portions and to secure the leg portions 104 and 106 to the wearer's body. The absorbent thigh regions 112 and 114 of leg portions 104 and 106 also serve as barriers by assisting in containing liquid which, for whatever reason, has not been absorbed by the central absorbent portion. In one aspect, the leg bands 146 include a circumferential elastic material around the leg openings. Other configurations of the leg bands can be provided, if desired.

The waist portion 102 should also fit snugly but comfortably against the wearer's body so as to maintain the garment's position against the wearer's body. In one aspect, the waist portion 102 can be provided with a waist band 118, which can be formed of a circumferential elastic material. In another aspect, the waist portion 102 can include, for example, one or more of snap, button, hook and loop fastener, and the like, to provide a close fit about the waist of the wearer. A loose fitting garment is more likely to result in the absorbent portions of the garment shifting such that the flow source is no longer in close proximity to the absorbent portions, which can increase the likelihood of leakage although the increased surface area of absorbent material of the garment described herein further reduces such risk of leakage.

Generally, the overall fit of the absorbent garment is snug such that the overall garment (or at least the absorbent portions thereof) is forced into close proximity to and into conformity with the wearer's body. The garment should expand and contract with the movement of the wearer's body and not sag or gap. Such a fit assists in preventing leakage of liquid by providing a barrier to the flow of liquid between the wearer's body and the garment. The garment should not fit so snugly as to interfere with bodily movements or provide discomfort to the wearer.

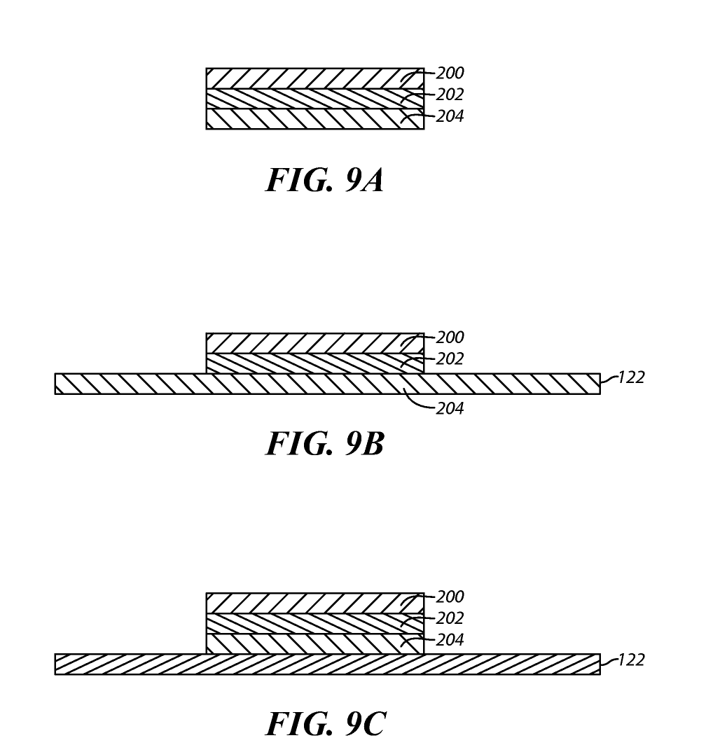

The absorbent portion of the garment may include any materials known in the art for the purpose of absorbing body fluids. As shown in FIG. 9A, for example, the absorbent portion can include a multi-layered absorbent structure, such as a three layer structure as diagrammed which includes a cover layer 200, an absorbent core layer 202, and a barrier layer 204, with the absorbent core layer 202 located between cover layers 200 and the barrier layer 204. The various layers can be secured together by any means known in the art, such as by gluing, adhesives, crimping, or heat sealing along the periphery of the absorbent portion to provide a seam. Of course, other mechanisms can be used, if desired, to unite the various layers of the absorbent portion.

The cover layer 200 (i.e., a top sheet) can comprise a liquid-permeable material which, when the garment is in use, is positioned adjacent the wearer's skin. Generally, this cover layer 200 comprises a woven or non-woven material or formed thermoplastic film that is soft and non-irritating (and possibly hypoallergenic if desired) to the wearer's skin. Suitable woven and non-woven materials include, for example, those made of natural fibers, synthetic fibers, or a combination thereof. The cover layer 200 is generally a substantially hydrophobic material. Natural fibers include, for example, cellulose fibers derived from wood pulp, cotton fibers, the like, and combinations thereof. Synthetic fibers include, for example, polymeric fibers such as polypropylene, polyethylene, polyester, nylon, rayon, the like, and combinations thereof. Preferred materials for the cover layer 200 are liquid pervious but non-absorbent so as to wick moisture from the skin. For washable garments, suitable materials known in the art for cover layer 200 include natural fibers, synthetic fibers, or a combination thereof. The cover layer preferably is a material that tends to wick moisture from the body and direct the moisture to the absorbent core 202.

In one aspect, the cover layer 200 may be treated with a hydrophilic surfactant to improve the wettability of the layer, which allows liquids to more easily penetrate the cover layer than with cover layers which do not include a surfactant. Generally, untreated cover layers allow fluids to flow quickly along the cover layer, which reduces the amount of time for the liquid to be absorbed by the absorbent core. However, the treated cover layer 200 should be less hydrophilic than the absorbent core layer for most application settings.

The barrier layer 204 is impermeable to liquid (i.e., menses or urine) under normal use conditions (where “normal” will be understood here in context to include high flow rates and/or slower flowing but high-volume flows that are not normal in the sense of being continuous and uninterrupted over time but which are nevertheless anticipated) so as to prevent liquids from leaking through the absorbent portion and soiling the wearer's outer garments. Depending on the desired configuration of the garment, the barrier layer 204 may be the same or different material as the material used for the exterior of the garment. As shown in FIG. 9B, the barrier layer 204 also serves as the exterior 122 of the garment while, in FIG. 9C, the barrier layer 204 is separate from the material which forms the exterior 122 of the garment. A variety of water impermeable materials may be used for the barrier layer, such as, but not limited to, polyethylene and polyurethane laminate (PUL).

The absorbent core layer 202 can comprise any of the numerous absorbent materials known in the art which include, but are not limited to, natural and synthetic materials. This absorbent core layer 202 can comprise one or more layers to provide the desired absorbence. For example, the absorbent core layer 202 can include cellulosic or fiber-based material, super absorbent polymers (SAP), the like, and combinations thereof. The absorbent core layer 202 can also utilize nanotechnology, if desired, such as through the use of nanoparticles as a nano-absorbent and/or through the use of nanotechnology to capture and retain moisture through the formation of a gel.

Generally, the absorbent core layer should be capable of absorbing at least 15 times its weight in 0.9% saline solution. In one aspect, the absorbent core layer as used herein should be capable of absorbing at least 25 times its weight in a 0.9% saline solution. In another aspect, an absorbent material should be capable of absorbing at least 30 times its weight in a 0.9% saline solution. In yet another aspect, an absorbent material should be capable of absorbing at least 35 times its weight in a 0.9% saline solution. Even higher absorbency materials can be used, if desired.

Cellulosic and fiber-based materials generally have reduced water-binding capacity as compared to SAP. SAP functions as a hydrogel by absorbing water through the formation of hydrogen bonds with water molecules. One exemplary SAP is sodium polyacrylate, although other polymers can be used as long as the polymers provide the desired absorbency for the garment. The absorbency of the SAP can depend on the level of crosslinking of the polymers. Generally, SAP with low density crosslinking provides higher absorbency and swelling capacity, while SAP with high density crosslinking provides lower absorbency and swelling capacity. Accordingly, the type of SAP selected for use in the absorbent garments described herein can be selected depending on the particular level of absorbency desired.

Polymeric absorbent structures can also be prepared such that the interior of the structure has low density crosslinking whereas there is a substantial degree of crosslinking at the surface of the structure such that the ability of water to leak through the surface of the polymeric structure is reduced while the interior of the structure maintains a high degree of absorbency. The type and degree of crosslinking of the SAP selected may also depend on the location of the absorbent material such that different areas of the garment may include materials having different levels of absorbency.

In one aspect, the fibers of the layers that comprise the undergarment are provided in a cross-hatched configuration. However, the exact orientation of the fibers can vary relative to the design of the panel and its integration into the undergarment. Accordingly, other configurations may be used if desired. Generally, the orientation of the fibers should increase the ability of the absorbent core to absorb liquids due to the capillary effect of the fibers.

Additional layers may also be included in the absorbent portion, if desired. For example, it may be desired to include an acquisition/distribution layer (often referred to in the art as “ADL”) between the core layer and cover layer. ADL can improve the comfort of the wearer, particularly by improving the feeling of dryness, by providing more rapid absorption of liquid by the core layer.

The level of absorbency provided throughout the different regions of the absorbent portion 126 may differ, for example, depending on the proximity to the flow source. By one approach, for example, the central absorbent portion of the garment can be provided with a higher absorbency material than that provided for absorbent thigh portions to accommodate greater amount of flow adjacent the flow source.

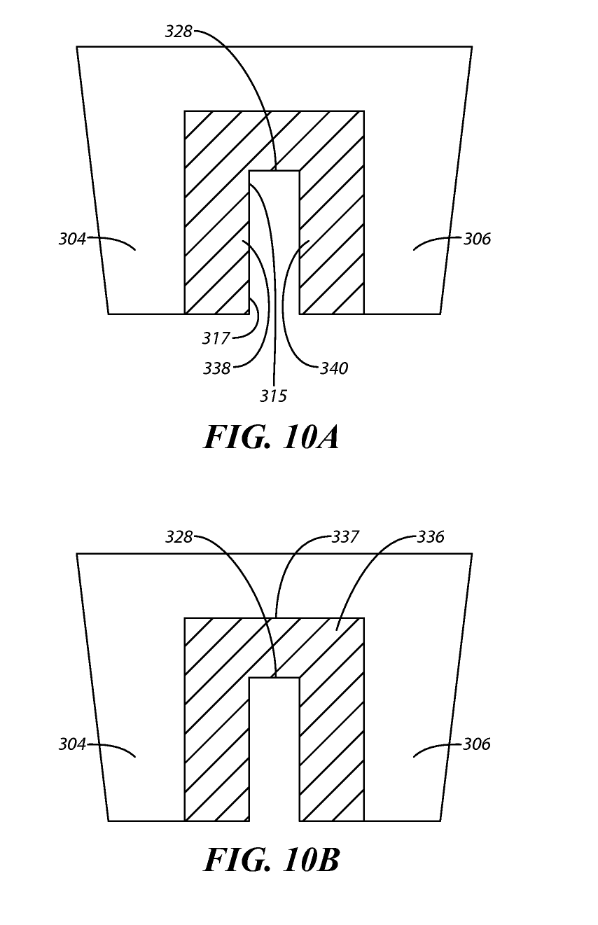

By another approach and as illustrated in FIG. 10(A), the leg portions 304 and 306 (including absorbent thigh regions 338 and 340) can be provided with greater absorbency towards the thigh portion inner end 315 adjacent the central absorbent region 328 (i.e., in close proximity to the flow source when the garment is worn by the user), while lower absorbency materials can be provided towards the thigh portion outer end 317, which is distal to the flow source when the garment is worn by the user. Of course, numerous other configurations of the various absorbencies can be provided as desired.

By another approach and as illustrated in FIG. 10(B), the absorbency of the forward and rear absorbent regions of the garment may vary depending on the specific needs of the wearer. In one aspect, the forward absorbent region 336 can be provided with greater absorbency towards the central absorbent region 328 (i.e., in close proximity to the flow source when the garment is worn by the user), while lower absorbency materials can be provided towards the upper end 337 of the forward absorbent region 336. The same teachings equally apply to rear absorbent region.

In one aspect, a gradient of high-absorbency material can be provided in any region of the absorbent portion. For example, high-absorbency material can be distributed in a non-uniform manner through the absorbent portion to form a gradient with either increasing or decreasing concentration of high-absorbency material as the proximity to the flow source increases. Alternatively, all regions of the absorbent portion 126 can be provided with the same level of absorbency if desired.

In another aspect, the thickness of the absorbent portion may vary. Referring generally to FIG. 7(B), for example, the thickness of the absorbent thigh regions 138 and 140 can decrease towards the leg bands 146. In another aspect, the thickness of the absorbent material in any of the absorbent portions can decrease or taper away from the flow source, such as decreasing in a horizontal direction away from the flow source toward the hips of the wearer or decreasing in a vertical direction upwards toward the waistband of the wearer.

The fabric forming the exterior of the garment can include a material that is comfortable yet has the flexibility required to provide the garment with a snug (i.e., closely conforming) fit. The terms “flexible,” “flexibility,” and similar terms refer to the property of readily conforming to the general shape and contours of the wearer's body. Suitable materials include natural and synthetic fabrics, such as, but not limited to, cotton and cotton blends, nylon, polyester, elastane, and combinations thereof.

The selection of the material for the exterior 122 and the barrier layer 204 may depend, at least in part, on whether the absorbent garment is a disposable or washable garment. For disposable garments, it may be more economical to provide at least a portion of the barrier layer 204 as the exterior of the garment. The term “disposable” means the garment is intended to be discarded after a single or limited use. The term “washable” means the garment is intended to washed, laundered, or otherwise put in condition for reuse as an absorbent garment. The barrier layer 204 can be given an appearance similar to that of cloth by the addition of a layer of polypropylene non-woven material to the barrier layer.

By one approach, the absorbent garment is provided in a disposable format such that the entire garment is intended to be discarded after a single or limited use.

By another approach, the absorbent garment is provided in a washable format such that all or portions of the garment are intended to be washed or laundered after each use. In one aspect, both the exterior of the garment and the absorbent portion of the garment can be provided with materials suitable for washing and laundering. In such a format, the absorbent portion(s) of the garment can be releasably attached to the interior of the garment. In another aspect, the exterior of the garment is provided in a washable format and the absorbent portion(s) of the garment is provided in a disposable format. By this approach, the absorbent portion(s) of the garment can be removed from the garment and replaced with a new absorbent portion(s) for each subsequent wearing.

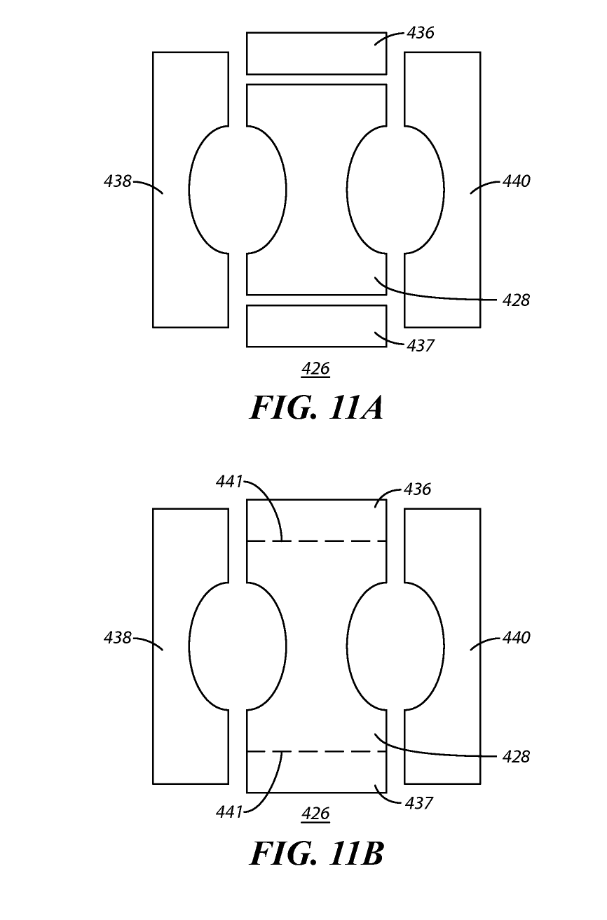

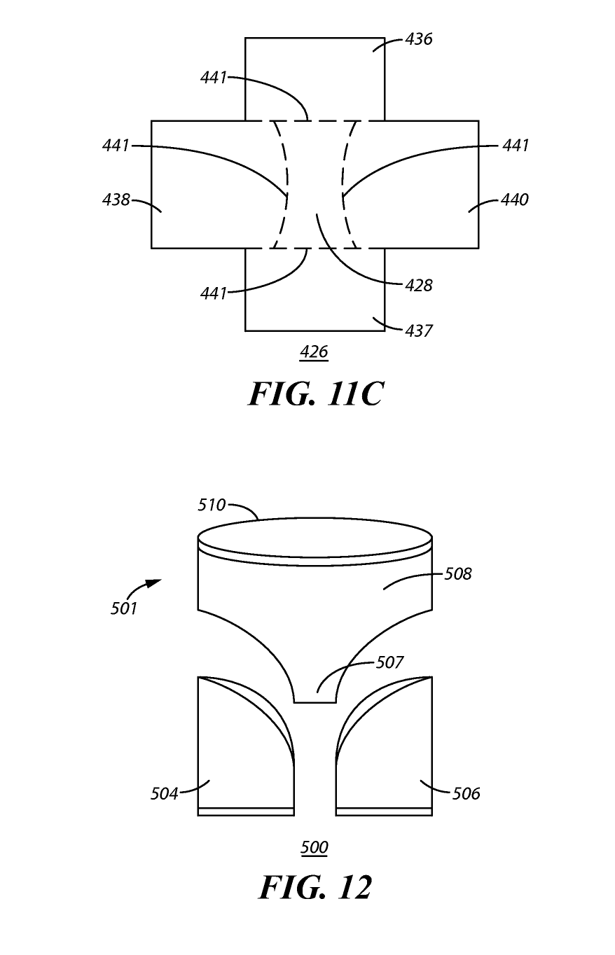

In either the washable or disposable formats, the absorbent portion(s) can be provided in separate sections such that individual sections of the absorbent portion(s) can be removed and replaced within the garment. Such an approach allows the user to replace soiled sections of the absorbent garment as needed. The sections can be provided in any desired configuration. One exemplary configuration of an absorbent portion is provided in FIG. 11(A), although numerous other configurations are contemplated and within the scope of the invention described herein. The absorbent portion 426 includes five separate sections, including one section each for the central absorbent region 428, two absorbent thigh regions 438 and 440, a forward absorbent region 436, and a rear absorbent region 437. Such sections can be assembled with a non-absorbent portion to provide an absorbent garment as is generally described above in reference to FIGS. 1-10.

A second exemplary configuration of an absorbent portion is provided in FIG. 11(B). Here, the absorbent portion 426 includes three separate sections, where the central absorbent region 428 is continuous with the forward and rear absorbent regions 436 and 437 to provide a single section which is separate from the absorbent thigh regions 438 and 440. The absorbent portion 426 may also include one or more seams 441. These seams 441 can provide points of weakness to the absorbent portion which provide increased flexibility so as to allow the absorbent portion to bend to fit a variety of sized garments as well as to better fit the contours of the wearer's body. These seams 441 may also allow for the detachment of the forward and rear absorbent regions 436 and 437.

By another approach and as pictured in FIG. 11(C), an absorbent portion 400 can be provided in a single part. The absorbent portion denoted by reference numeral 426 includes the same regions already described herein, which include the central absorbent region 428, two absorbent thigh regions 438 and 440, the forward absorbent region 436, and the rear absorbent region 438. This absorbent portion 426 may also include one or more seams 441 which allow for bending the absorbent portion to better fit the contours of the wearer's body. These seams 441 may also allow for the detachment of the absorbent thigh regions 438 and 440, as well as the forward and rear absorbent regions 436 and 437.

The absorbent portions presented in FIG. 11(A)-(C) are not shown to scale but are merely intended to show non-exhaustive options for configurations of the various regions of the absorbent portion. Other configurations can also be used, if desired.

In regard to the configurations presented in FIG. 11(A)-(C), the absorbent portion 426, provided in one or more sections, can be inserted into and attached to the interior of a short or pant garment, skort, long underwear, hosiery, or the like to provide an absorbent garment in accordance with the teachings presented herein. The type of garment in which the absorbent portion is inserted is not intended to be limiting but the garment should be the type of garment that has material which contacts the inner thigh of the wearer such that the absorbent inner thigh regions of the absorbent portion can be attached thereto. The sections of the absorbent portion 426 can be attached using conventional techniques, such as, but not limited to, snaps, buttons, clips, hook and loop fasteners, adhesives, and the like. The number of sections used to provide the absorbent portion 426 is not believed to be particularly important provided that the sections are assembled such that there is not leakage between the assembled sections.

By yet another approach and as an alternative to the format provided in FIG. 1(A), an absorbent garment 500 is pictured in FIG. 12. Absorbent garment 500 can be provided in three sections: a main body portion 501, a right leg portion 504, and a left leg portion 506. The main body portion 501 includes a front torso portion 508, a back torso portion 510, and a crotch portion 507. The main body portion 501, a right leg portion 504, and a left leg portion 506 can be connected to one another via conventional techniques as previously described. All or a portion of the main body portion, right leg portion 504, and left leg portion 506 can be provided with absorbent material as previously described herein and with reference to FIGS. 3-11.

For formats where all or a section of the absorbent portion is intended to be releasably attached to the garment, such removable section(s) are provided with mechanisms by which the removable sections can be adhered to the garment. Such mechanisms may include, for example, an adhesive backing, hook and loop fasteners, or any other mechanism known in the art. In one aspect, the underside of the absorbent portion (i.e., the side of the absorbent portion not intended to contact the skin) is at least partially coated with an adhesive. A removable silicon-coated paper can be used to temporarily cover the adhesive prior to use.

In one aspect, the absorbent garment described herein provides a similar function as traditional undergarments, such as briefs, panties, hosiery, or boy-shorts, such that the garment can be worn without additional undergarments.

In one aspect, the absorbent garment is provided in the form of a “boy-short” or “brief” format. Boy-short or briefs are particularly preferred because, unlike panties, these garments provide coverage of the buttocks and inner thigh regions of the body. Longer shorts or pants can also be used to provide an absorbent garment as described herein, if desired. Other style undergarments can also be used with the absorbent portion described herein provided that the undergarments allows the absorbent thigh portions of the undergarment to be provided with a snug fit about the inner thighs.

By one approach, the garment can be provided in a variety of sizes to accommodate a variety of body shapes and sizes. For example, the exact, length, and width of the waist, leg portions, front and back torso portions, and leg openings can vary depending on the dimensions of the intended wearer. In one aspect, the absorbent garment is designed so that the garment is not bulky and has a smooth appearance such that the garment is not readily detectable under the wearer's outer garments.

In another aspect, the absorbent garment may further comprise additives which mask the scent of a soiled garment and/or soothe the skin and prevent or treat chafing and redness. Exemplary additives include, but are not limited to, perfumes, balms, emollients, fragrance, creams, and oils. These teachings would also accommodate intermixing charcoal particles in at least some portions of such a garment to potentially neutralize at least some corresponding odors. It may also be desired that the materials used for washable formats of the absorbent garment (i.e., garments not intended for single use) be made with stain resistant fibers or materials.

The absorbent garment described herein is intended for use as a personal care or personal hygiene product to absorb bodily fluids from a flow source. The absorbent garment is not intended to serve as an athletic garment for wicking sweat from the body or to provide cushioning or padding to the body during athletic activities. While the absorbent garment may incidentally provide cushioning or padding to the body, the primary function and materials used to provide the absorbent garment is to protect the wearer against leakage of bodily fluids and to prevent soiling of the wearer's outer garments.

Those skilled in the art will recognize and appreciate that a wide variety of modifications, alterations, and combinations can be made with respect to the above described embodiments without departing from the spirit and scope of the invention, and that such modifications, alterations, and combinations are to be viewed within the ambit of the inventive concept.

What is claimed is:

1. An absorbent garment comprising a crotch portion, a waist portion, and two leg portions, the crotch portion including a central absorbent region having a first surface area of absorbent material, the two leg portions each comprising an absorbent thigh region, the absorbent thigh regions having a combined second surface area of absorbent material, the combined second surface area of absorbent material of the thigh regions being at least the same as the first surface area of the central absorbent region.

2. The absorbent garment of claim 1, wherein the combined second surface area of the absorbent this regions is at least 10 percent greater than the surface area of the central absorbent region.

3. The absorbent garment of claim 1, wherein the combined second surface area of the absorbent this regions is at least 20 percent greater than the surface area of the central absorbent region.

4. The absorbent garment of claim 1, wherein each leg portion has a circumference of 360 degrees and at least about 90 degrees of the circumference comprises absorbent material.

5. The absorbent garment of claim 1, wherein the central absorbent region includes a center point and the central absorbent region extends about 2 to about 7 inches forward of the center point.

6. The absorbent garment of claim 5, wherein the central absorbent region includes a center point and the central absorbent region extends about 2 to about 7 inches rear of the center point.

7. The absorbent garment of claim 1, wherein each absorbent thigh region is configured to extend at least about 1 inch downward from the central absorbent region.

8. The absorbent garment of claim 1, wherein each absorbent thigh region is configured to extend at least about 2 inches downward from the central absorbent region.

9. The absorbent garment of claim 1, wherein each absorbent thigh region is configured to extend at least about 3 inches downward from the central absorbent region.

10. The absorbent garment of claim 1, wherein each leg portion has a circumference of 360 degrees and about 90 to about 180 degrees of the circumference comprises absorbent material.

11. The absorbent garment of claim 10, wherein the about 130 degrees of absorbent material is configured to contact the wearer's inner thigh area.

12. The absorbent garment of claim 10, wherein each leg portion includes about 130 to about 270 degrees of absorbent material.

13. The absorbent garment of claim 1, wherein the absorbent material comprises a multi-layered absorbent structure, including a cover layer, an absorbent core layer, and a barrier layer.

14. The absorbent garment of claim 13, wherein the absorbent core layer comprises super absorbent polymers.

15. The absorbent garment of claim 1, wherein at least one of the central absorbent region and the absorbent thigh regions are configured to be detached from the garment.

16. An absorbent garment comprising a comprising a crotch portion, a waist portion, and two leg portions, the crotch portion including a central absorbent region, the two leg portions each comprising an absorbent thigh region, the central absorbent region including a center point and the central absorbent region extending about 2 to about 7 inches forward of the center point, and each absorbent thigh region is configured to extend at least about 2 inches downward from the central absorbent region.

17. The absorbent garment of claim 16, wherein each leg portion has a circumference of 360 degrees and at least about 90 degrees of the circumference comprises absorbent material configured to contact the wearer's inner thigh area.

18. An absorbent portion configured to be inserted into an undergarment, the absorbent portion comprising:
a central absorbent region having a first surface area of absorbent material;
two absorbent thigh regions, each inner thigh absorbent region located on opposite sides of the center absorbent region, the absorbent thigh regions having a combined second surface area of absorbent material, the combined second surface area of absorbent material of the thigh regions being at least the same as the first surface area of absorbent material.

19. The absorbent portion of claim 18, wherein each absorbent thigh region is configured to extend at least about 2 inches from the central absorbent region.

20. The absorbent portion of claim 18, wherein each absorbent thigh region is configured to extend at least about 3 inches from the central absorbent region.
